(19) United States
(12) Patent Application Publication
(10) Pub. No.: US 2014/0303502 A1
(43) Pub. Date: Oct. 9, 2014

(54) METHOD AND APPARATUS FOR MEASURING HEARTBEATS WITHOUT CONTACT AND WIRELESSLY

(71) Applicant: Electronics and Telecommunications Research Institute, Daejeon (KR)

(72) Inventors: Yong Kwi LEE, Daejeon (KR); Sangwook PARK, Daejeon (KR); Hyun Jin YOON, Daejeon (KR); Ji Yeon KIM, Daejeon (KR); Jong Hyun JANG, Daejeon (KR)

(73) Assignee: Electronics and Telecommunications Research Institute, Daejeon (KR)

(21) Appl. No.: 14/203,480

(22) Filed: Mar. 10, 2014

(30) Foreign Application Priority Data
Apr. 5, 2013 (KR) 10-2013-0037474
Oct. 31, 2013 (KR) 10-2013-0131372

(51) Int. Cl. A61B 5/024

(52) U.S. Cl. CPC A61B 5/02416
USPC 600/474

(57) ABSTRACT

Provided is a method of measuring heartbeats without contact and wirelessly. The method includes searching for a sport point on which heartbeats are measured; imaging a found spot point through a thermal graphic camera and obtaining temperature related data wirelessly; and analyzing the obtained temperature related data and calculating heartbeats.

### CROSS-REFERENCE TO RELATED APPLICATIONS

This U.S. non-provisional patent application claims priority under 35 U.S.C. §119 of Korean Patent Application Nos. 10-2013-0037474, filed on Apr. 5, 2013, and 10-2013-0131372, filed on Oct. 31, 2013, the entire contents of which are hereby incorporated by reference.

### BACKGROUND OF THE INVENTION

The present invention disclosed herein relates to a method of measuring heartbeats wirelessly, and more particularly, to a method and apparatus for measuring the heartbeats of a subject without contact and wirelessly by measuring the temperature of skin in which an artery/ vein lies, by using a thermal graphic camera.

With the recent developments of imaging devices, smart phones and wireless communication technologies, apparatuses that have been typically connected in a wired manner are being connected wirelessly.

In general, since e.g., a biological signal is weak, it is difficult to measure the signal wirelessly.

On the other hand, when the biological signal is measured in a wired manner, a subject needs to be in an awkward state and thus experiences inconvenience.

### SUMMARY OF THE INVENTION

The present invention provides a method of measuring heartbeats wirelessly.

The present invention also provides an apparatus and method that may continue to image a part of a body by using a thermal graphic camera and may thus measure heartbeats wirelessly, without measuring heartbeat signals in a wired manner.

Embodiments of the present invention provide methods of measuring heartbeats without contact and wirelessly, the method including searching for a spot point on which heartbeats are measured; imaging a found spot point through a thermal graphic camera and obtaining temperature related data wirelessly; and analyzing the obtained temperature related data to calculate heartbeats.

In some embodiments, the spot point may be a point on which a carotid artery or a jugular vein lies.

In other embodiments, the calculating of the heartbeats may be performed by counting how many times the skin temperature varies for a preset time.

In still other embodiments, the spot point may be a part distant by 3 Cm to 5 Cm inwards from the edge of a neck and a part distant by 2 Cm to 3 Cm downwards from a chin.

In other embodiments of the present invention, apparatuses for measuring heartbeats without contact and wirelessly include a thermal graphic camera imaging a spot point found from a subject, the thermal graphic camera obtaining temperature related data wirelessly; and a computer analyzing the obtained temperature related data and measuring heartbeats.

In some embodiments, the computer may include a measurement controlling unit receiving data on skin temperature varying whenever heart of the subject pounds, the measurement controlling unit counting how many times the skin temperature varies for a preset time; and a memory storing the data on the skin temperature and the count result.

In still other embodiments of the present invention, apparatuses for measuring heartbeats without contact and wirelessly include a thermal graphic sensing unit imaging a spot point found from a subject, the thermal graphic camera obtaining thermal graphic sensed data wirelessly; a camera control unit allowing the thermal graphic sensing unit to sense the spot point and converting the thermal graphic sensed data into temperature related data; a communication interface outputting the temperature related data and receiving control data; and a measurement controlling unit analyzing the temperature related data and measuring heartbeats.

In even other embodiments of the present invention, methods of measuring heartbeats of an animal without contact and wirelessly include searching for a sport point on which heartbeats are measured; imaging a found spot point through a thermal graphic camera and obtaining temperature related data wirelessly; and counting, from the obtained temperature related data, a number of variations in skin temperature varying whenever the heart of the animal pounds, and measuring the heartbeats of the animal.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a schematic diagram of an apparatus for measuring heartbeats according to an embodiment of the present invention;

FIG. 2 is a flowchart of a method of measuring heartbeats by using FIG. 1;

FIG. 3 is a circuit block diagram of a thermal graphic camera in FIG. 1; and

FIG. 4 is a circuit block diagram of a computer in FIG. 1.

### DETAILED DESCRIPTION OF PREFERRED EMBODIMENTS

The above objectives, other objectives, characteristics and advantages of the present invention will be easily understood through the following embodiments to be described with reference to the accompanying drawings. However, the present invention is not limited embodiments to be described below but may be implemented in other forms. Rather, these embodiments are provided so that this disclosure will be thorough and complete, and will fully convey the scope of the present invention to those skilled in the art, for the purpose of providing only the convenience of understanding.

In the specification, when some elements or lines are referred to as being connected to a target element block, it should be understood that the former can be directly connected to the latter, or indirectly connected to the latter via another element.

Moreover, the same or like reference numerals in each of the drawings represent the same or like components if possible. In some drawings, the connection of elements and lines is just represented to effectively explain technical content and may further include other elements or circuit blocks.

An embodiment described and exemplified herein includes a complementary embodiment thereof, and it should be noted that details of fundamental processing on image data and internal software are not described in order not to obscure the subject matter of the present invention.

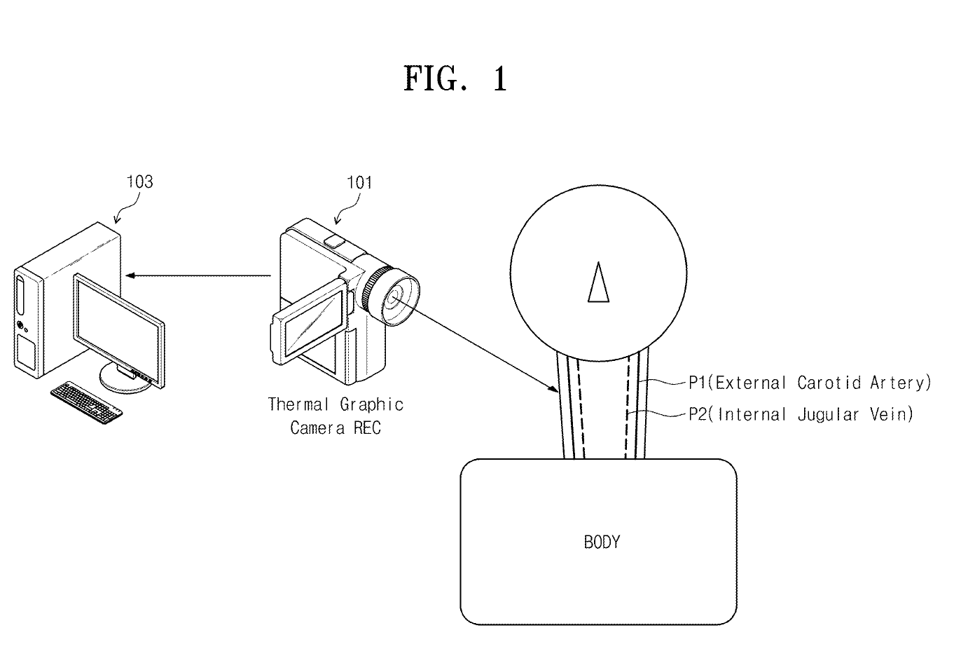

FIG. 1 is a schematic diagram of a system for measuring heartbeats according to an embodiment of the present invention.

A thermal graphic camera 101 is a thermal infrared camera, images a spot point found from a subject, and obtains temperature related data wirelessly.

A computer 103 analyzes the temperature relayed data and measures heartbeats.

The thermal graphic image 101 focuses on the external carotid artery P1 or the internal jugular vein P2 of a neck 102 of a patient and measures the temperature of skin. In this case, in order to measure heartbeats for a certain time, imaging is performed without departing from the spot point for the certain time (such as one minute).

An obtained video image is the temperature related data and is transmitted to the computer 103. The computer 103 analyzes how many times the temperature of skin varies per minute, by using the obtained video image.

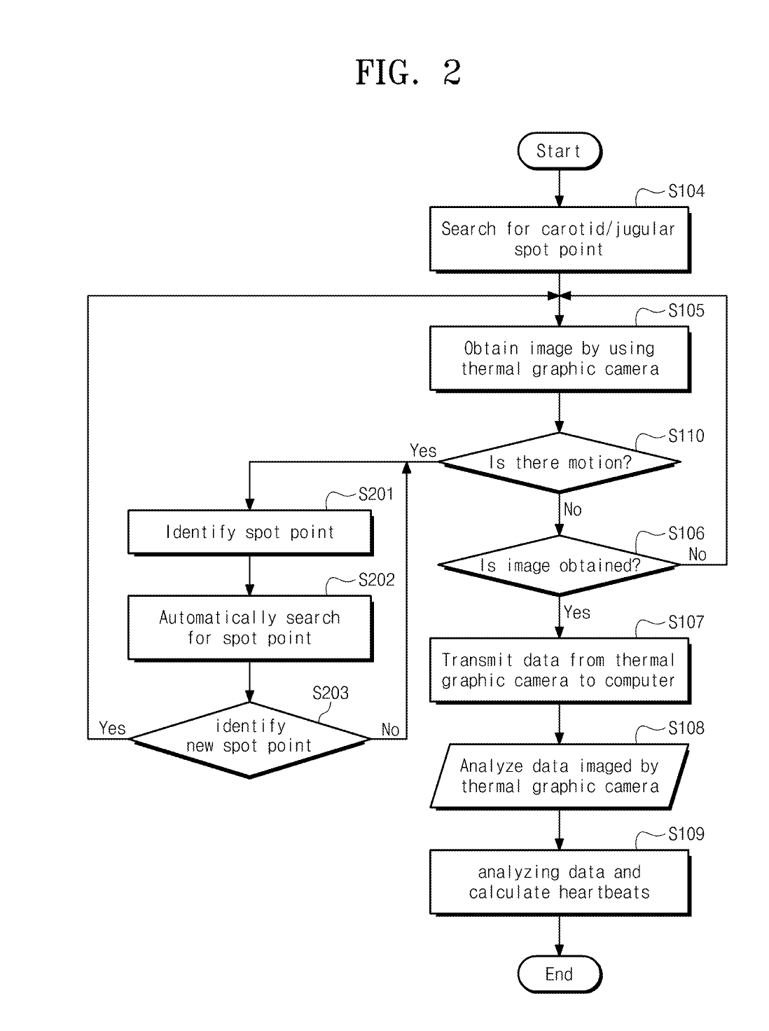

The temperature of blood spouted from the heart of an animal or a human being is different from that of capillary in general skin. That is, whenever heart pounds, the temperature of skin in which arterial blood flows rises. Thus, when continuing to image an artery part as the spot point, it is seen that the temperature of skin slightly rises if heart pounds. Thus, if counting how many times the temperature of the spot point varies per minute, it is possible to know heartbeats per minute. In this example, the one minute is only exemplary and the time may vary. FIG. 2 is a flowchart of a method of measuring heartbeats by using FIG. 1.

Referring to FIG. 2, in order to perform the method of measuring the heartbeats wirelessly, the thermal graphic camera 101 needs to search for carotid artery and jugular vein spot points. The spot points of the carotid artery and the jugular vein may be generally a part distant by 3 Cm to 5 Cm inwards from the edge of a neck and a part distant by 2 Cm to 4 Cm downwards from a chin. When the thermal graphic camera focuses on the spot point, it is possible to see through the system for measuring the heartbeats that the temperature of skin varies whenever heart pounds.

In step S104, when the spot point is found, the thermal graphic camera 101 starts imaging the spot point in step S105.

In this case, when there is a change in the spot point due to the motion of a subject in step S110 while imaging, the automatic search and tracking of the carotid artery and the jugular vein are performed. The automatic search and tracking of the carotid artery and the jugular vein are performed when the spot points depart by a certain value from a part distant by 3 Cm to 5 Cm inwards from the edge of a neck and a part distant by 2 Cm to 4 Cm downwards from a chin. As a result, when there is a change in the spot point due to motion, the spot points of the carotid artery and the jugular vein are again searched through steps S201 to S203 and heartbeats are accurately measured.

In step S202, when the spot point departs by a value equal to or larger than ±2 Cm from an initial spot point on which imaging has been performed, automatic search and tracking start. A distance is calculated by using the infrared ray of the thermal graphic camera 101. As a result, when the spot point departs by a value equal to or larger than ±2 Cm from an initial spot point, the initial spot point is tracked by a backward search using the value ±2 Cm in step 5203. After step 5203, step S105 is again performed.

Automatic tracking on the spot point is performed by using such a principle so that imaging is performed on a constant spot point.

The thermal graphic camera 101 determines whether imaging is performed for a certain time in step 5106. If negative, the thermal graphic camera 101 continues to image, and if positive, it ends imaging.

In step 5107, information imaged by the thermal graphic camera, namely, the temperature related data is saved as a file and transmitted to the computer 103.

In step 5108, the computer 103 opens the file transmitted from the thermal graphic camera and counts how many times the temperature of skin in the image varies for a certain time (such as one minute).

As such, when the counted number of variations in the temperature of skin is associated with one minute, it becomes the final measurement result of heartbeats per minute.

On the other hand, when an image obtained for a time shorter than or equal to one minute, for example, when the number of variations in the temperature of skin is counted for ten seconds, six is multiplied to know heartbeats per minute. As a result, since six is multiplied, heartbeats per minute are obtained through step S109. It should be noted that although the present invention describes that heartbeats are measured for one minute, a measuring time period is not limited thereto and may increase or decrease.

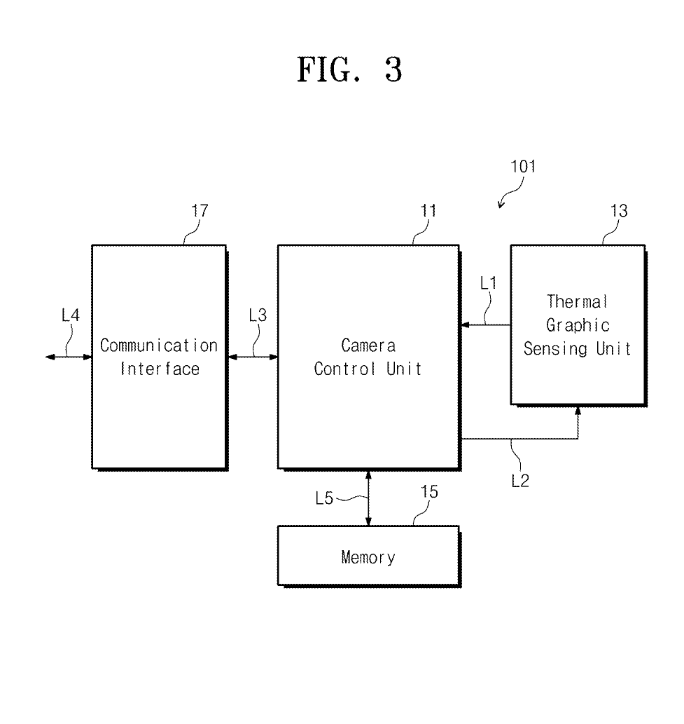

FIG. 3 is a circuit block diagram of a thermal graphic camera in FIG. 1.

Referring to FIG. 3, the thermal graphic camera includes a camera control unit 11, a thermal graphic sensing unit 13, a memory 15, and a communication interface 17.

The thermal graphic camera 13 images the spot point found from a subject and obtains thermal graphic sensing data wirelessly.

The camera control unit 11 controls a line L2 so that the thermal graphic sensing unit senses the spot point, receives the thermal graphic sensing data through a line L1, and converts the received data into temperature related data.

The communication interface 17 outputs, through a line L4, the temperature related data received through a line L3 and receives control data applied through the line L4.

The communication interface 17 may be configured to communicate with an internal or external device through at least one of various interface protocols such as a universal serial bus (USB) protocol, a multimedia card (MMC) protocol, a peripheral component interconnection (PCI) protocol, a PCI-express (PCI-E) protocol, an advanced technology attachment (ATA) protocol, a serial-ATA protocol, a parallel-ATA protocol, a small computer small interface (SCSI) protocol, an enhanced small disk interface (ESDI) protocol, and an integrated drive electronics (IDE) protocol.

The memory 15 is connected to the camera control unit 11 though a line L5 and stores image data. The memory 15 may be a video RAM.

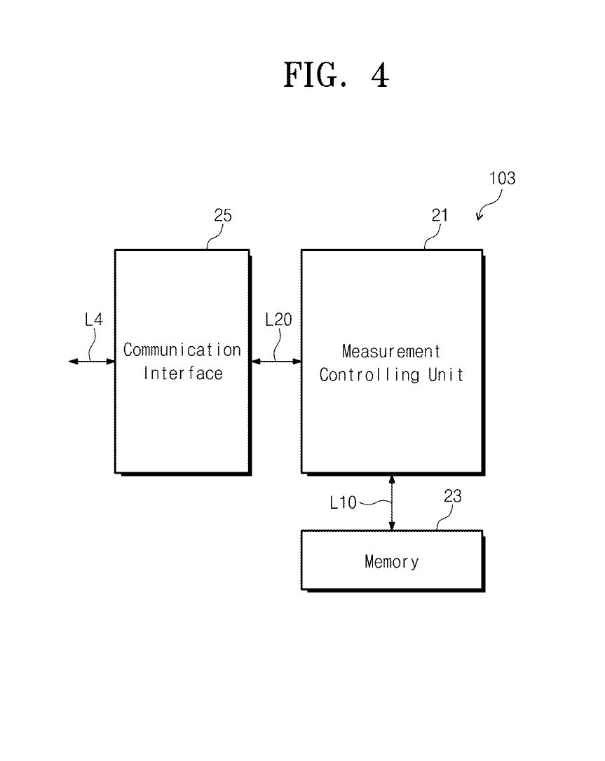

FIG. 4 is a circuit block diagram of a computer in FIG. 1.

Referring to FIG. 4, the computer 103 includes a measurement control unit 21, a memory 23, and a communication interface 25.

The measurement control unit that may be implemented in a micro processor receives, through the communication interface 25 connected to a line L20, data on skin temperature varying whenever the heart of the subject pounds, and counts how many times the skin temperature varies for a preset time.

The memory 23 connected to the measurement control unit 21 through a line L10 stores data on the skin temperature and a count result.

The memory 23 may be implemented in a volatile semiconductor memory device such as a DRAM or an SRAM, or a non-volatile semiconductor memory device.

The non-volatile semiconductor memory device may be implemented in an electrically erasable programmable read-only memory (EEPROM), a flash memory, a magnetic RAM (MRAM), a spin-transfer torque MRAM, a conductive bridging RAM (CBRAM), a ferroelectric RAM (FeRAM), a phase change RAM (PRAM) referred to also as an ovonic unified memory (OUM), a resistive RAM (RRAM or ReRAM), a nanotube RRAM, a polymer RAM (PoRAM), a nano floating gate memory (NFGM), a holographic memory, a molecular electronics memory device), or an insulator resistance change memory, for example. In an embodiment of the present invention, the computer 103 may be one of an ultra mobile PC (UMPC), a workstation, a net-book, a personal digital assistant (PDA), a portable computer, a web tablet, and a tablet computer.

Since wireless heartbeat measurement according to the present invention adopts a technique of wirelessly measuring a variation in skin temperature unlike a typical technique of measuring pulses on a finger, there is no need to attach a measurement element to a body.

As a result, it is possible to measure heartbeats by imaging a spot point, so heartbeats are measured without contact and awkwardness. Thus, when measuring heartbeats, user convenience increases and it is possible to measure heartbeats easily and conveniently in daily life or in a hospital.

As described above, while particular embodiments have been described in the detailed description of the present invention, several variations may be made without departing from the scope of the present invention. Therefore, the scope of the present invention should not be limited to the above-described embodiments but be defined by the following claims and equivalents thereof.

For example, also when measuring heartbeats of an animal in stead of a human being, it is possible to vary a spot point. Also, the functions of the computer may be implemented by incorporating a circuit for measuring heartbeats into the thermal graphic camera.

Also, by employing a wireless interface in the thermal graphic camera, it is also possible to transmit heartbeats data to other electronic devices connected to the internet through WiFi or other communication networks.

According to the heartbeats measurement of the present invention, it is possible to wirelessly measure heartbeats through imaging without measuring a biological signal in a contact technique. Thus, since the heartbeats of a subject are measured without contact and awkwardness, use convenience increases. Therefore, it is easy and convenient to measure heartbeats even without significantly limiting the movement of a subject when the subject is in daily life or in a hospitable.

Also, since it is possible to measure the heartbeats of an animal by only imaging without attaching equipment to the body of the animal that is difficult to measure heartbeats, the present invention may be used in various fields such as veterinary medicine.

What is claimed is:

1. A method of measuring heartbeats without contact and wirelessly, the method comprising:
searching for a spot point on which heartbeats are measured;
imaging a found spot point through a thermal graphic camera and obtaining temperature related data wirelessly; and
analyzing the obtained temperature related data to calculate heartbeats.

2. The method of claim 1, wherein the spot point is a point on which a carotid artery or a jugular vein lies.

3. The method of claim 1, wherein the thermal graphic camera is a thermal infrared camera.

4. The method of claim 1, wherein the temperature related data is data on a skin temperature varying whenever heart pounds.

5. The method of claim 4, wherein the calculating of the heartbeats is performed by counting how many times the skin temperature varies for a preset time.

6. The method of claim 4, wherein the calculated heartbeats are externally transmitted through a wireless network or remotely transmitted through a wired network.

7. The method of claim 1, wherein the calculating of the heartbeats is performed by sensing a variation in temperature rising whenever heart pounds.

8. The method of claim 1, wherein the spot point is an internal jugular vein or an external carotid artery.

9. An apparatus for measuring heartbeats without contact and wirelessly, the method comprising:
a thermal graphic camera imaging a spot point found from a subject, the thermal graphic camera obtaining temperature related data wirelessly; and
a computer configured to analyze the obtained temperature related data and to measure heartbeats.

10. The apparatus of claim 9, wherein the subject is a human body.

11. The apparatus of claim 9, wherein the spot point is a point in which a carotid artery or a jugular vein lies.

12. The apparatus of claim 9, wherein the computer comprises:
a measurement controlling unit receiving data on skin temperature varying whenever heart of the subject pounds, the measurement controlling unit counting how many times the skin temperature varies for a preset time; and
a memory storing the data on the skin temperature and the count result.

13. The apparatus of claim 12, wherein the time preset by the measurement controlling unit varies depending on the subject.

14. The apparatus of claim 12, wherein the computer internally outputs or transmits through a communication network, information on heartbeats obtained according to the count result.

15. The apparatus of claim 12, wherein the thermal graphic camera senses a variation in temperature whenever the heart of a human body pounds.

16. The apparatus of claim 12, wherein the measurement controlling unit is a microprocessor measuring heartbeats according to a preset program.

17. The apparatus of claim 12, further comprising a communication interface connected to the measurement controlling unit and performing data communication with the outside in a wired manner or wirelessly.

18. An apparatus for measuring heartbeats without contact and wirelessly, the apparatus comprising:
a thermal graphic sensing unit imaging a spot point found from a subject, the thermal graphic camera obtaining thermal graphic sensed data wirelessly;
a camera control unit allowing the thermal graphic sensing unit to sense the spot point and converting the thermal graphic sensed data into temperature related data;
a communication interface outputting the temperature related data and receiving control data; and
a measurement controlling unit analyzing the temperature related data and measuring heartbeats.
